(19) United States
(12) Patent Application Publication
(10) Pub. No.: US 2010/0260721 A1
(43) Pub. Date: Oct. 14, 2010

(54) Enrichment of Cells

(75) Inventors: Dennis McGonagie, Leeds (GB); Elena Jones, Leeds (GB); Anne English, Leeds (GB); Paul Emery, Leeds (GB)

Correspondence Address: LERNER, DAVID, LITTENBERG,;KRUMHOLZ & MENTLIK, 600 SOUTH AVENUE WEST, WESTFIELD, NJ, 07090, US

(73) Assignee: University of Leeds, Leeds (GB)

(21) Appl. No.: 12/223,056

(22) PCT Filed: Jan. 17, 2007
(86) PCT No.: PCT/GB2007/000107

§ 371 Date: Jul. 18, 2008

(30) Foreign Application Priority Data
Jan. 18, 2006 (GB) 0600972.4

(51) Int. Cl. A61K 35/48; C12N 5/0735; A61P 19/10; A61P 19/02

(52) U.S. Cl. 424/93.7; 435/372

(57) ABSTRACT

The present invention relates to methods of isolating and enriching mesenchymal stem cells (MSCs) comprising treating a tissue sample comprising cells and extracellular matrix with an amount of a collagenase that is sufficient to free MSCs from the extracellular matrix in a liquid medium and then isolating a fraction of the medium containing MSCs. The isolated MSCs can be isolated in surprisingly large numbers such that the fraction can have immediate use in a number of clinical contexts. This represents an advance of prior art techniques that either require the pooling of large volumes of sample tissues from different sources or require MSCs to be expanded in culture. The invention therefore further concerns the clinical use of cells isolated according to the methods of the first aspect of the invention.

The present invention relates to the enrichment of mesenchymal stem cells (MSC).

MSCs are considered important cells that may be used in a number of scientific and clinical applications. For instance, MSCs have been proposed, and investigated, for use in cell-based therapies.

MSCs are useful because they are pluripotent stem cells that can be induced to differentiate into a number of cell types including bone, cartilage and fat cell lineages. MSCs may be induced to differentiate in vitro and then used as “mature” cells or may be maintained in undifferentiated form and allowed to differentiate in situ. MSCs also have the useful characteristic that they are able to proliferate as adherent cell monolayers in vitro.

Unfortunately MSCs have to date had limited use scientifically and clinically. One reason for this is that it has been difficult to readily isolate useful numbers of MSCs. For instance, bone marrow is known to be a source of MSCs and a number of researchers have attempted to isolate such cells from bone marrow aspirates. However only small numbers of cells have been isolated and this has led the scientific community to believe that only very small numbers of MSCs exist in such tissues. This represents a problem because impractical volumes of tissue would be required to isolate clinically useful numbers of MSCs.

The only way of generating useful numbers has been to isolate MSCs and then culture them in vitro, sometimes for several weeks, in order that the numbers may be expanded or “bulked-up”.

However such culturing steps have a number of disadvantages.

First, culture expansion is time consuming. This limits the clinical usefulness of MSCs. There are some instances where there is an acute need for stem cells. For instance, if a clinician wished to administer stem cells to a subject with a broken bone to encourage healing it would be desirable to have a suitable number of stems cells available within hours of the injury. However conventional wisdom would dictate that the clinician should isolate MSCs from bone marrow aspirates from the injured subject; then expand the population in culture; and after some time introduce the stem cells to the injured bone. However the whole process may take several weeks and, after this time, the MSCs may be of little use in the partly healed tissue. Accordingly many cell-based therapies are impractical because a source of cells is not readily available.

Second, the culture of MSCs can lead to “phenotypic drift” in the cultured cells. This drift may arise should the cultured MSCs begin to differentiate. This differentiation can be unpredictable and can often be along an undesired lineage. Alternatively the artificial culture conditions may prevent the cells from differentiating to a phenotype that would be possible if the cells had remained in vivo. It is therefore recognized that cultured cells may not grow the same as cells would in vivo. This also leads to the clinical and scientific community questioning the value of MSCs expanded in culture.

Third, the manipulation and expansion of MSCs in culture may lead to senescence of MSCs that could contribute to subsequent loss of function in vivo. Prolonged culture expansion of MSCs could also lead to genetic instability and cell transformation. Furthermore, regulatory bodies such as the FDA have questioned the advisability of using animal products in the generation of cells for therapeutic human use as is the case for cell culture expansion procedures for MSCs.

Finally, it is often difficult to successfully achieve ex vivo expansion of stem cell populations. In order to overcome this difficulty, it is frequently necessary to use exogenous factors such as serum and growth factors in order to promote stem cell proliferation. Such agents may be both costly and lead to complex culture conditions requiring the efforts of skilled technicians.

It will therefore be appreciated that it would be desirable to secure an immediate, convenient source of MSCs that do not have to be expanded in vitro. However, to date there is no readily available source of such cells and it is one object of the present invention to provide such as source of MSCs.

According to a first aspect of the present invention there is provided a method of enriching mesenchymal stem cells (MSCs) comprising:
(a) treating a tissue sample comprising cells and extracellular matrix with an amount of a collagenase that is sufficient to free MSCs from the extracellular matrix in a liquid medium; and
(b) isolating a fraction of the medium containing MSCs.

It is preferred that step (b) involves isolating the MSCs using tight phenotypic criteria as discussed in more detail below.

According to a second aspect of the present invention there is provided isolated mesenchymal stem cells (MSC) enriched according to the first aspect of the invention.

According to a third aspect of the present invention there is provided isolated mesenchymal stem cells (MSC) enriched according to the first aspect of the invention for use as a medicament.

According to a fourth aspect of the invention there is provided a kit for enriching mesenchymal stem cells (MSC) comprising a collagenase and optionally media for maintaining, growing or differentiating the cells.

By the term “Mesenchymal Stem Cell” we mean pluripotent or multipotent stem cells that are able to proliferate as adherent cell monolayers and may be induced to differentiate into cells of mesodermal lineages (e.g. bone, cartilage, muscle, tendon, ligament or fat cells). Suitable cues for the differentiation described above may be provided by environmental factors including soluble factors (such as growth factors), and the substrate on which the stem cells or their progeny are located. Pluripotent and multipotent are taken to have their conventional meanings, i.e. that pluripotent cells are stem cells capable of differentiating to give rise to several differentiated lineages; whereas multipotent cells may comprise progenitor or precursor cells with more limited differentiation potential, i.e. able to give rise to diverse cell types within the same lineage.

Mesenchymal stem cells in the context of the present invention may include stem cells derived from bone marrow cavity (including both aspirates and cores of trabecular bone), synovial membranes or fat pads (as discussed further below). For example, mesenchymal stem cells in the context of the present invention may include stem cells of the type that have previously been isolated from processed lipoaspirates (LPAs) and identified as having the capacity to give rise to lineages including osteoblasts, chondrocytes, myocytes, adipocytes and neuron-like cells.

The present invention is based upon the work described in Examples 1, 2 and 3 in which the inventors established that surprising numbers of MSCs may be enriched from bone marrow cavity, synovium and joint fat pads respectively. They established that the enrichment step may comprise a relatively simple enzymatic tissue digest according to the first aspect of the invention. This step was surprisingly found to yield a population of MSCs with the phenotype: CD45^{low}D7-FIB⁺LNGFR⁺. D7-FIB is a monoclonal antibody (available commercially from suppliers such as Serotec, Novus Biologicals or Abcam) that recognizes fibroblast/epithelial cells, and LNGFR is the low-affinity nerve growth factor receptor, CD271, (available commercially from suppliers such as Becton Dickinson or Miltenyi Biotec).

The inventors demonstrated that this CD45^{low}D7-FIB⁺LNGFR⁺ cell population comprised clonogenic and pluripotent MSCs. This lead them to realise that routine clinical tissue material could be used as a source of sufficient numbers of MSCs (around 10⁶ of highly purified cells) to be used directly for a variety of important therapeutic, diagnostic and research purposes. This was considered very surprising because preparations isolated by conventional methods only yield a small number of MSCs that need that to be expanded or “bulked-up”, with all the associated disadvantages discussed above, to form useful numbers. The surprising numbers of MSCs yielded by collagenase treatment of a tissue sample enables a clinician or a scientist to immediately have available useful numbers of cells that may be used without exposure to the artificial environment of in vitro cell culture.

The tissue sample from which the MSCs are enriched according to the invention may be derived from a number of sources that would be well known to one skilled in the art to comprise MSCs. These include bone marrow, trabecular bone (both seen as integral parts of the same organ, bone marrow cavity) and also soft tissues such as synovium and joint fat pads. The present invention does not relate to the fact that MSCs would, or would not, be expected to be found in such tissues. For instance it is well known that bone marrow comprises MSCs and such MSCs have been isolated, in very small numbers, from bone marrow aspirates. These cells need to be isolated from the aspirates and cultured to bulk-up MSC numbers to scientifically or clinically useful numbers. The inventive step of the present invention lies in the fact that collagenases may be used to liberate fresh MSCs from tissues (e.g. a biopsy sample containing both bone marrow and trabecular bone) in unprecedented numbers. It could not be predicted from the prior art that scientifically and/or clinically useful numbers of fresh MSCs were (i) contained in relatively small volumes of tissue; and (ii) could be liberated by enzymatic treatment. In fact there was a technical prejudice against attempting to enrich MSCs according to the present invention. This is because a skilled person would have expected such a low yield of cells that the volume of tissue requiring to be processed to yield useful numbers of MSCs would have been impractical. This common opinion is based on a historical perception of an MSC being a rare cell at the apex in the mesenchymal progenitor cell hierarchy; whereas the inventors consider that a high degree of plasticity, consistent with current MSC definition, is inherent to many connective tissue cells, including some pre-committed progenitors and mesenchymal lineage perivascular cells (pericytes).

It is most preferred that step (b) of the method of the invention involves selecting a population of MSCs by applying strict phenotypic criteria. According to the state of the art there is: (1) a lack of sufficient knowledge on the phenotype of MSCs in vivo, hence a lack of an ability to clearly discriminate between MSCs and contaminating cells; (2) a lack of realization that fresh MSCs may be clinically useful because freshly extracted MSCs have higher proliferative potential compared to culture-expanded MSCs—accordingly fewer “fresh” cells may be required than would be expected by the skilled person; and (3) a lack of appreciation that even partially-pure but fresh MSC preparations (a fraction isolated according to step (b) may contain platelets, macrophages and other growth factor-producing cells) may be more clinically useful than phenotypically homogenous culture-manipulated MSCs.

Phenotypic selection/identification of MSCs may be based on identifying a number of MSC markers on the target cell population. These markers may include those identified in table 1.

TABLE 1

| Marker/ | Other | Reactivity with |
|---|---|---|
| Mab | Names | Fresh MSCs |
| CD45 | CLA | Low |
| Gly A | Glycophorin A | − |
| D7-FIB | NA | +++ |
| CD271 | LNGFR, NGFR, p75 | ++++ |
| CD13 | Aminopeptidase N | +++ |
| CD73 | SH3 | +++ |
| CD105 | SH2 | + |
| CD90 | Thy-1 | +++ |
| CD63 | HOP-26 | ++ |
| CD49a | Intergin VLA-1 | ++ |
| CD118 | LIF receptor | ++ |
| CD130 | Gp130 | ++ |
| CD81 | TAPA-1 | +++ |
| CD39 | ATPDase | +++ |
| JAM2 | VEJAM | + |
| CD10 | CALLA, MME | ++ |
| ALP | Alkaline phosphatase | +++ |
| CD146 | MCAM | + |

The inventors found, although many markers were useful, that certain markers were particularly useful for identifying fresh MSCs prepared according to the invention. Accordingly D7-FIB, LNGFR (i.e. CD271), CD13, CD73 and CD90 are particularly useful markers. Most preferred markers for identifying fresh MSCs identify cells with the phenotype: CD45^{low}D7-FIB⁺LNGFR⁺. The inventors consider LNGFR⁺ to be the single most important marker for identifying MSCs according to the invention.

It will be appreciated that the abovementioned markers are useful for isolating MSCs according to step (b) of the invention as-well-as for the purposes of identifying the cells. For instance, as discussed in more detail below, the inventor's knowledge of MSC phenotype may be exploited when isolating MSCs by FACS sorting.

It is preferred that the tissue sample is bone marrow (BM) or a core biopsy containing both BM and trabecular bone (TB). Methods of enriching MSCs from these tissues are described in more detail below, and in Example 1. The invention is based on the inventors' work characterising the phenotype and demonstrating low yields of MSCs (based on this phenotype) recovered from BM aspirates. They established that all MSC activity in BM aspirates was confined to a rare population of CD45^{low}D7-FIB⁺LNGFR⁺ cells. Accordingly, anybody wishing to isolate MSCs, should select CD45^{low}D7-FIB⁺LNGFR⁺ cells. The Example describes how they further investigated the frequency and clonogenicity of this population of cells. It was during these investigations that they were pleasantly surprised to find that phenotypically pure MSCs had many properties in common with stromal adventitial reticular cells (ARCs). BM stromal network consists of ARCs, endothelial cells and surrounding pericytes, adipocytes, macrophages and endosteal cells, the latter cells lining the bone surfaces. ARCs form an interconnected network of cells involved in haemopoiesis-supportive stromal function and their role as precursors of adipocytes and osteoblasts has been suggested. The inventors hypothesised that MSC activity and plasticity is inherent to many if not all ARCs, pericytes, pre-adipocytes and endosteal cells and that all these cells belong to the same mesenchymal cell lineage. The inventors' data on the similar phenotype of ARCs and MSCs supported this hypothesis. The fact that fairly specialized mesenchymal cells (such as adipocytes or osteoblasts) can trans-differentiate and de-differentiate in vitro is described in the literature. Hence the inventors suggested that similar processes may occur in vivo and that harvesting these mesenchymal lineage cells (which can be only achieved by enzymatic treatment) would liberate increased numbers of highly-plastic MSCs. In the opposite, during BM aspiration these mesenchymal cells remain in situ, being tightly attached to each other and to a surrounding extracellular matrix (in comparison to loosely-attached haemopoietic cells which are easily aspirated). The inventors postulated therefore that low numbers of MSCs previously found in BM aspirates were not at all representative of their actual numbers in vivo. Accordingly, they found that unprecedented numbers of CD45^{low}D7-FIB⁺LNGFR⁺ cells (MSCs) could be liberated from tissues by treating tissue samples with a collagenase according to the method of the first aspect of the invention. This lead them to appreciate that scientific and clinical useful quantities of CD45^{low}D7-FIB⁺LNGFR⁺ cells/MSCs could be quickly and easily obtained from small volumes of tissue.

MSCs can also be isolated according to the invention from other connective or soft tissues. These tissues include the synovium and joint fat pads (as discussed in Examples 2 and 3 respectively).

The inventors have demonstrated that the methods exploited with bone marrow biopsy samples may be applied to these tissues to enrich surprising numbers of MSCs with the same basic phenotype and similar functionality (i.e. CD45^{low}D7-FIB⁺LNGFR⁺). The inventors have also recognised that CD45^{low}D7-FIB⁺LNGFR⁺ cells resident in different tissues may be defaulted to specific local differentiation pathways and hence be useful for different applications (i.e. BM CD45^{low}D7-FIB⁺LNGFR⁺ cells will be more suitable for bone repair applications whereas synovial CD45^{low}D7-FIB⁺LNGFR⁺ cells will be more suitable for cartilage repair applications). The inventors showed that in the synovium and fat pads, CD45^{low}D7-FIB⁺LNGFR⁺ cells have the topography of pericytes (i.e. mesenchymal cells involved in supporting tissue vascularisation and neovascularisation). They showed that these CD45^{low}D7-FIB⁺LNGFR⁺ occupy the same niche as vascular pericytes, are probably identical; are clonogenic; and have similar functionality as the BM MSCs. Hence the method of the invention provides a means of enrichment of MSCs from this particular perivascular niche in solid tissues including synovium and fat. The method of the invention allows purification of most of bone marrow MSC activity and also allows for the purification of most synovial and fat pad MSC activity.

The method of the first aspect of the invention can be used for isolation of MSCs from the perivascular (pericyte) niche from other tissues, including but not restricted to, placenta and umbilical cord. These tissue sources are considered to be rich for MSCs particularly suitable for neurodegenerative applications. Accordingly MSCs enriched from placenta or umbilical cord, according to the method of the invention, may preferably be used in the treatment of neurodegenerative conditions. Isolated soft tissue MSCs in a pericyte distribution, not only have the same phenotype as BM MSCs, but may have all of the potential of BM MSCs and hence can be used in all applications discussed below.

The source of the tissue sample will depend upon the ultimate use of the enriched MSCs.

A preferred tissue sample is from bone and in particular bone marrow (BM). Enriched MSCs from BM samples are particularly useful for bone repair applications. BM biopsy material may be obtained from a number of body sites (e.g. the sternum, femur, pelvis or iliac crest). Preferred sources of BM are femoral heads or the iliac crest. Specimens may be collected by punch biopsy (e.g. using a 4-mm punch biopsy). Alternatively, femoral heads can be cut in half and homogenized using Bone Mill (De Puy or other manufacturers) to obtain even smaller fragments to undergo enzymatic digestion. Autologous BM may be obtained from iliac crest biopsy from patients or volunteers and larger numbers of MSCs could be obtained from any trabecular bone in cadaveric donors for research purposes or for allogenic MSC applications.

Prior to collagenase treatment, core biopsy samples or small tissue fragments may be placed in PBS (phosphate-buffered saline) to prevent them from drying. However sample should not be washed extensively with PBS. This is to preserve BM so that MSCs traversing the marrow (ARCS) and MSCs attached to bone (endosteal cells) are both available for enzymatic extraction.

In a preferred embodiment the tissue sample on which enrichment in accordance with the present invention is to be effected may comprise the floating fat fraction (FFF) derived from bone marrow. The inventors have found this fraction, which has previously been discarded during the isolation of MSCs, to constitute a surprisingly rich source of MSCs.

By way of example only, 0.1 grammes of tissue sample (taken as a single biopsy) may be placed in 0.5 mls of a collagenase solution (e.g. a commercial collagenase solution from Stem Cell Technologies (product number 07902), which is a 0.25% collagenase solution in 20% (v/v) fetal bovine serum (FCS)).

It will be appreciated that the collagenase may be obtained from a number of commercial sources (e.g. Stemcell technologies Inc, Vancouver, CA). A number of different collagenases may be used. However it is preferred that the enzyme is effective for digesting Collagen I.

It will be appreciated that an amount of collagenase will be required that is sufficient to liberate MSCs from the sample being treated. The amount will depend on the number of factors including: the type of sample used (i.g. a sample from a bone biopsy or fat pad); the amount of sample being treated; the volume of the buffer/solution in which the sample is contained; and the incubation time with the enzyme. As a general guidance, the inventors have found that between about 100 and 500 Units of enzyme, and more preferably between 250 and 375 Units of enzyme, is sufficient for liberating MSCs from a 0.1 g BM biopsy sample, synovium or fat pad sample (in a 0.5 ml volume, at 370 C for an incubation time of 3-4 hours).

Furthermore 100 and 500 Units of enzyme, and more preferably between 250 and 375 Units of enzyme, is sufficient to liberate about 10,000-50,000 MSCs and more preferably about 25,000-150,000 MSCs (purified to 100% purity by FACS) from such a 0.1 g BM sample. It is surprising and significant that such significant numbers of MSCs can be liberated because similar, and even smaller doses, of fresh MSCs are known to have efficacy in children with OI and in the treatment of bone non-unions. It will be appreciated that there will be more cells in a preparation if only “semi-pure” MSCs are required (e.g. a cell fraction decanted off the collagenase treated sample which has not undergone a further enrichment step e.g. further purification using microbeads or FACS sorting).

Incubation of the sample should be conducted at the optimal temperature for collagenase activity. This is normally about 37° C. However it will be appreciated that some collagenases have slightly different optimal temperatures for catalysis and/or may be able to operate over a range of temperatures (e.g. 20° C.-47° C.).

In a preferred embodiment of the method of the first aspect of the invention a 4 mm punch biopsy BM sample is placed in 0.5 ml of 0.25% collagenase. The enzyme may be left, at 37° C., to digest the sample for 0.5-8 hours but more preferably for 3-4 hours.

During incubation, an hourly inspection and “tapping” of the sample may be performed, to monitor the release of cells from a biopsy. Following release of cells, collagenase solution becomes cloudy whereas BM biopsy itself becomes whiter (exposing bone) or soft tissue biopsy fully dissolves. Specifically for BM biopsy samples, a second separate 1-hour collagenase treatment may be preferable to release bone-lining cells and hence obtain cell fraction especially enriched for MSCs with the highest osteogenic potential. At the end of the preferred incubation time, a final tapping may be performed to ensure maximal cell release.

Collagenase activity may be stopped using a number of procedures known to the art (e.g. dilution, filtration or centrifugation followed by decanting). A preferred means of stopping collagenase activity and isolating the cells according to step (b) comprises: diluting the sample with an over 20-fold excess of PBS (v/v) or similar buffer. A syringe/needle may be used to mix the released cells with PBS and to enforce remaining cells to egress from the biopsy into the solution. Immediately after, PBS/cell mixture may be sieved through 70 micron cell strainer and may then be transferred into another centrifuge tube for centrifugation (5 minutes at 1800 rpm). Collagenase/PBS solution is then discarded and the cell pellet is re-suspended in media appropriate for cell counting and subsequent manipulations (magnetic and FACS separation), usually DMEM with 2% FCS.

Following collagenase digestion and cell isolation (e.g. as described in the preceding paragraph), the inventors have found that the proportion of phenotypically pure MSC population (CD45^{low}″D7-FIB⁺LNGFR⁺ cells) is over 100-fold higher than that in aspirates. The number of cells may be 5-10% of the total cells released or more. Such preparations are clinically useful according to the invention.

In some embodiments of the invention, the inventors have established that optimal MSC preparations may be prepared by including further enzymatic digestions in step (a). It will therefore be appreciated that the choice of further enzymatic digestions will be dictated by the tissue sample being treated and by the ultimate use of the MSCs. By way of example the inventors have found that a second collagenase treatment and/or a hyluronidase treatment can be particularly useful for liberating the maximum number of MSCs for bone. Protocols for conducting a second collagenase treatment and hyluronidase treatment are described in the Examples. It is most preferred that step (a) is adapted to include a second collagenase treatment and/or a hyluronidase treatment when the tissue is bone and the MSCs will be used for the purposes of bone repair.

According to certain embodiments of the invention, step (b) may be supplemented by further procedural steps that may be applied to improve the quality of MSC preparations and these may involve (1) removal of dead cells/debris and (2) further enrichment for MSCs.

The removal of cell debris/dead cells may be achieved by a number of known procedures (e,g. using Dead Cell Removal Kit from Miltenyi Biotec). This step is desirable if further enrichment is to be achieved using magnetic beads (see below). However the inventors have found that this removal of cell debris/dead cells is not required if the further enrichment step is FACS sorting (also see below).

For subsequent magnetic-based enrichment of MSCs from the released cell fraction several approaches may be used. For instance the inventors carried out positive selection with Anti-Fibroblast microbeads (directly conjugated with D7-FIB antibody), MACSelect microbeads (directly conjugated with LNGFR antibody) or CD271-APC microbeads (all from Miltenyi Biotec, Bisley, UK). Both methods led to a subsequent over 10-fold increase in the MSC proportion (to 50-60% of total cells in collagenase digests). Based on the inventors knowledge of the phenotype of MSCs, they believe that other microbeads that may be useful are CD73-conjugated or CD105-conjugated (the latter is commercially available from Miltenyi Biotec). Alternatively lineage-negative depletion methods can be used that utilize carefully selected antibody cocktails that definitely do not cross-react with MSCs. Therefore the use of the present invention with the use of microbeads could lead to at least a 10 fold greater availably of MSCs for therapy development than was hitherto thought to be possible.

FACS sorting may be used to further enrich the MSC fraction produced according to the method of the invention. MSC purity of over 95% can be easily achieved by FACS sorting. Furthermore dead cells/debris can be eliminated based on PI, 7AAD or other dead cell exclusion stains. FACS sorting allows MSC purification to be achieved in less than one hour.

It will be appreciated that MSC enrichment may also be achieved by utilising antibodies or other agents that will recognise cell surface markers on the cells. Table 2 provides examples of antibodies, and the markers they recognise, that may be used to isolate MSCs.

TABLE 2

| | Other | | |
|---|---|---|---|
| Mab | Names | Conjugate | Source |
| CD45 | CLA | FITC | DAKO, BD |
| D7-FIB | NA | Purified | Serotec |
| CD271 | LNGFR, NGFR, p75 | PE | BD, |
| | | PE, APC | Miltenyi Biotec |
| CD13 | Aminopeptidase N | PE, FITC | Serotec |
| CD73 | SH3 | PE | BD |
| CD105 | SH2 | PE | Serotec |
| CD90 | Thy-1 | FITC | BD |
| CD63 | HOP-26 | PE | BD |
| CD49a | Intergin VLA-1 | PE | BD |
| CD118 | LIF receptor | PE | BD |
| CD130 | Gp130 | PE | BD |
| CD81 | TAPA-1 | PE | BD |
| CD39 | NA | FITC | BD |
| JAM2 | NA | PE | Serotec |
| CD10 | CALLA, MME | APC | BD |
| ALP | Alkaline phosphatase | PE | Developmental Studies |
| | | | Hybridoma Bank, |
| | | | University of Iowa |
| CD146 | MCAM | PE | BD |

Fractions comprising the MSCs may be used immediately. Alternatively the MSCs may be frozen, using conventional cryogenic techniques for use at a future date.

MSCs purified according to the first aspect of the invention are extremely useful to scientific investigators in the MSC and stem cell field in general.

A skilled person will appreciate that the methods, cells and kits according to the invention are also particularly useful in a variety of prognostic or diagnostic tests. For instance, haematology laboratories may enrich MSCs (according to the invention)/bone marrow stromal supportive cells as a part of a diagnostic test to screen for stromal abnormalities in diseases including, but not restricted to, myelofibrosis, myeloma and myelodysplastic syndrome. Bone/orthopaedics/rheumatology/endocrinology and other clinical departments may enrich for MSC/osteoblast progenitors as a part of a diagnostic test to screen for osteoblast abnormalities in osteoporosis, osteopetrosis, osteoarthritis and osteonecrosis. Diagnostic tests for diabetes/obesity may utilise MSC/fat cell progenitors/preadipocytes, enriched according to the invention, as a part of a diagnostic test to screen for abnormalities in fat cell development in such diseases. Furthermore diagnostic and prognostic tests concerning “ageing” may utilise MSCs enriched according to the invention as a part of a diagnostic test to screen for/predict their potency for autologous therapy prior to implantation.

The method of the invention will be particularly useful to scientists elucidating the biology of the bone marrow microenvironment. The method provides a way of purifying large numbers of stromal supportive cells allowing their biology to be studied ex vivo in health and disease. Bone marrow stromal cells/MSCs undergo considerable changes following culture expansion making current Dexter type stromal cultures unreliable surrogates for in vivo biology. This invention will allow the bone marrow microenvironment to be better understood in normal marrow, myelodsyplastic bone marrow and malignant bone marrow. The ability to study the marrow stroma ex vivo could have major implications for defining growth factors, chemokine and cytokine support of an array of primary bone marrow disorders, eg multiple myeloma and metastatic bone marrow diseases. This could lead to an array of new therapies for bone marrow disorders. Likewise the potential for understanding the bone marrow microenvironment in health and disease could have implications for the development of new therapies for the treatment of bone related diseases including osteoporosis.

The method of the invention may be employed to produce medicaments according to the third aspect of the invention that may be used in cell therapy techniques. Cell therapy can utilise both autologous and allogenic MSCs.

Medicaments according to the third aspect of the invention should comprise cells isolated according to the method of the first aspect of the invention and a physiologically acceptable buffer or scaffold. The buffer may comprise a sterile cell culture medium (and preferably a defined medium that does not require the addition of serum or the like) that maintains MSCs in a viable state and which also is safe for administration to a subject to be treated. The scaffold may comprise a sterile biocompatible material that supports MSC attachment and differentiation and which also is safe for administration to a subject to be treated.

MSCs provide according to the first aspect of the invention are particularly useful when autologous cell therapy is required. The method of the first aspect of the invention enables a clinician to obtain a sample of tissue from a subject; treat the tissue with collagenase to liberate clinically useful number of MSCs; and then re-introduce the cells to the subject being treated. The method of the invention allows the clinician to rapidly re-introduce MSCs to the subject from which the tissue sample has been taken. For instance, the cells could be re-introduced within a few days, on the same day or, with careful co-ordination of clinical and support staff within hours of taking the sample (e.g. within 3 hours). This may mean that tissue samples may be taken and enriched MSCs re-introduced without needing to remove the subject from the treatment room or operating theatre. This represents a significant improvement over prior art techniques which involved harvesting the MSCs; culturing the MSCs to bulk-up numbers; and then re-introducing the cells to the subject (usually at least several weeks later). This represents a particularly important feature of the invention. Therefore according to a fifth aspect of the invention there is provided a method of performing cell therapy on a subject in need of such therapy comprising:
(a) obtaining a tissue sample comprising MSCs and extracellular matrix from said subject;
(b) treating said tissue sample with an amount of a collagenase that is sufficient to free MSCs from the extracellular matrix into a liquid medium;
(c) isolating a fraction of the medium containing MSCs; and
(d) re-introducing said fraction into said subject at a body site that permits said subject to benefit from said cell therapy.

The inventors have found that MSCs may be isolated from a subject in surprising numbers that are sufficient for re-introduction into the same subject in therapeutically relevant quantities.

It is preferred that MSCs may be enriched (e.g. from the bone marrow/trabecular bone) according to the fifth aspect of the invention by employing the methods of the first aspect of the invention.

It is preferred that the fraction is re-introduced within 24 hours, preferably within 12 hours and preferably within 3-4 hours of obtaining the sample.

It is preferred that the cells used according to the method of the fifth aspect of the invention are used such that there is no need to freeze said MSCs.

The inventors have established that the invention is applicable to a number of autologous cell therapy regimens including, but not restricted to:
(1) Bone repair (traumatic bone fracture, segmental bone defect, craniofacial reconstruction, sinus lift indication, spine fusion indication, bone tumour/cyst indication)
(2) Cartilage repair (traumatic injury, osteoarthritis)
(3) Skeletal muscle repair (Duchenne Muscular Dystrophy and others)
(4) Tendon repair (traumatic injury in athletes)
(5) Ligament repair (traumatic injury in athletes)
(6) Meniscus repair (traumatic injury in athletes)
(7) Cardiac muscle repair (following myocardial Infarction and cardiomyopathies)
(8) Spinal cord injury, Parkinson's diseases and other neurodegenerative diseases
(9) Immune-mediated tissue rejection, graft-versus-host disease and rheumatoid arthritis (as immunomodulatory cell therapy)
(10) Regeneration of fatty material (e.g. reconstitution of fat pads associated with the metatarsals or the like; or regeneration of fatty tissue associated with breast, for instance in breast remodeling after surgery or accident).
(11) MSCs may differentiate into endothelial cells. Accordingly the method is useful useful for vascular surgery or ischaemic vascular lesions where very large numbers of cells capable of forming endothelium may be required in the treatment.
(12) Osteoporosis.

It is particularly preferred embodiment of the invention MSCs prepared according to the present invention are utilised in bone repair (1 above). Over one million orthopaedic operations annually involve bone repair as a consequence of replacement surgery, trauma, cancer, osteoarthritis, osteoporosis, congenital abnormalities or skeletal deficiency. Reconstruction of large bone defects continues to require bone grafts, which have many disadvantages, including donor site damage, pain and potential risks of infection and pathogen transmission. In osteoporosis, total hip replacement surgery remains the only treatment currently available for displaced hip fractures. Even with new advances in bone anabolic drugs (such as teriparatide and strontium ranelate) that decrease the risk of new fractures, the worldwide incidence of osteoporotic hip fracture is projected to increase a minimum 3-fold in the next 50 years as the population ages. Finding new ways of repairing these fractures, and healing bone in general, therefore is an urgent healthcare priority. In this context, cell therapies with MSCs offer a new solution.

MSCs are adult stem cells lack ethical concerns associated with embryonic stem cells and are known to form bone in vivo. Accordingly bone tissue engineering with MSCs (optionally with osteoconductive scaffolds and osteoinductive growth factors) is an alternative strategy to bone allo- and autografts in trauma and reconstructive surgery. For fracture treatment, dramatic improvements in the rates of bone union and the quality of repaired bone were achieved by local delivery of MSCs. MSC cell therapy is also a very useful treatment of osteogenesis imperfecta (OI).

To date these known treatments have relied upon the expansion of MSCs in culture in order that clinically useful numbers may be generated. Although MSC culture-expansion yields almost limitless supply of cells, the manufacturing process is long and costly. This precludes or strictly limits access to these products for the general public (e.g. for government funded health-care such as the NHS in the United Kingdom). In addition to high cost, extended MSC culture is associated with serious biological problems, such as potential for cell transformation, accumulation of mutations, cell senescence and loss of multipotentiality. Finally, important regulatory issues still exist for the assessment of purity and osteogenic “potency” of expanded MSCs. For example, CD73, CD105 and other “classic” markers of cultured MSCs have now been proven not to be specific for multipotent cells (expressed on skin and other types of fibroblasts). Therefore a niche for new, cost-effective and better-characterised MSC products exists.

It will be appreciated that MSCs prepared according to the method of the first aspect of the invention will fill this niche. The inventors have surprisingly found that fresh MSCs can be isolated from tissue samples in clinically useful numbers. In contrast to expanded MSCs, a clear advantage of freshly isolated MSCs is the speed at which this cell product can be obtained. According to the method of the first aspect of the invention, a small trabecular bone biopsy sample can yield ˜5×10⁵ pure MSCs (CD45^{low}″D7-FIB⁺LNGFR⁺ cells) following a 5-hour isolation procedure. A second advantage of freshly isolated MSCs is their full phenotypic characterisation showing high purity. A third advantage is their higher potential for osteogenesis.

In a further preferred embodiment of the invention MSCs prepared according to the present invention are utilised in cartilage repair (2 above) and in particular for treating osteoarthritis (OA). OA is a disease characterised by joint decomposition with joint organ failure. The loss of articular cartilage is thought to be a key primary event in a subgroup of sufferers. Therapeutic intervention with small molecules has no proven role in cartilage regeneration. The current gold standard for cartilage repair utilising cellular therapy is the autologous chondrocyte implantation (ACI) procedure. However conventional ACI can take months to complete from the time of initial cartilage harvesting to therapy. This is a very expensive procedure and consequently availability is limited. Furthermore this procedure is only suitable for younger subjects as cells from cartilage show an age related decline in proliferation and differentiation thus contributing to a lack of success with the ACI procedure in older subjects. Also the procedure is potentially detrimental since it involves taking a biopsy from the cartilage in the first instance. Nevertheless, the ACI procedure is the benchmark against which all other cellular therapies will be judged. Recently ACI related developments including the use of matrices to assist cellular engraftment have been developed but these have not circumvented the wider limitations of the procedure. In comparison to bone regeneration where a number of bone inducing matrices, growth factors and cellular products have already reached the marketplace the situation is rather different for cartilage repair.

It will therefore be appreciated that MSCs, which have the potential to differentiate into articular cartilage, could be viewed as a potential source of cells for therapy development in OA in a technique reminiscent of the ACI procedure. However, the perceived rarity of MSCs means that these cells must also go through lengthy culture expansion procedures with the attendant risks of infection, exposure to fetal calf serum, cell senescence, loss of potency, expense and of cost. The methods of the present invention enable a clinician to rapidly obtain large numbers of stem cells and quickly return these to the OA joint cartilage defect. This represents a significant progress in the therapy of OA. An ability to rapidly isolate large numbers of MSCs is of particular relevance in the present climate given that chondral defects are being increasingly recognised with the widespread use of magnetic resonance imaging for the assessment of OA.

In a still further preferred embodiment of the invention MSCs prepared according to the present invention are utilised in the treatment of osteoporosis. The inventors have noted that the biology and phenotype of MSCs (prepared according to the invention), which contain the osteoprogenitors, is considerably different from culture expanded MSCs. Culture expanded MSCs have formed the gold standard for the assessment of osteoprogenitor function in man. The ability to perform large scale purification of MSCs/osteoprogenitors could open up new avenues for therapeutic pathway discovery in osteoporosis.

According to a further embodiment of the invention, the inventors have established that MSCs may also be isolated, according to the invention, from a foreign source and then utilised in cell therapy in a subject suffering from a medical condition that may be treated by stem cell therapy (e.g. a bone fracture or OA). Allogenic MSCs may be enriched from cadaveric donors from various sites including hip, vertebreal and sternal marrow; relatives/siblings; or matched unrelated donors. Therefore accordingly to a sixth aspect of the invention there is provided a method of performing cell therapy on a subject in need of such therapy comprising:
(a) obtaining a tissue sample comprising MSCs and extracellular matrix from a cadaver or donor;
(b) treating said tissue sample with an amount of a collagenase that is sufficient to free MSCs from the extracellular matrix in a liquid medium;
(c) isolating a fraction of the medium containing MSCs; and
(d) introducing said fraction into said subject at a body site that permits said subject to benefit from said cell therapy.

It will be appreciated that the cadaver or donor is of the same species as the subject. It is most preferred that the subject is human. However, the inventors have noted that the LNGFR marker is preserved in a number of other animals (e.g. cows, horses, goats and sheep). It will therefore be appreciated that the same procedure could therefore be used to obtain cells for bone or cartilage repair in animals (e.g. pets such as dogs and particularly horses).

The enriched allogenic source of MSCs may be used in the same therapeutic regimens (1-12) listed in connection with the fifth aspect of the invention.

Tissues from which MSCs are to be enriched in accordance with the methods of the invention may be selected with reference to desired therapeutic properties to be achieved on subsequent differentiation of the cells. For example, the inventors have found that MSCs enriched from synovial tissues give rise to cells having notable chondrogenic properties, and accordingly it may be preferred that MSCs be enriched from synovium in situations in which it is desired to therapeutically promote the production of cartilage.

Similarly, the inventors have found that MSCs enriched from infrapatella fat pads give rise to cells having consistently high adipogenic properties, and accordingly it may be preferred that MSCs be enriched from fat pads in situations in which it is desired to therapeutically promote the production of adipose tissue.

MSCs enriched in accordance with the sixth aspect of invention may also find applications in gene therapy under the circumstances where the allogenic MSCs have a genotype that will correct a genetic defect in the subject being treated. Conditions that may be treated with such cells include:
(a) Congenital abnormalities associated with the loss of bone and cartilage function, including, but not restricted to, osteogenesis imperfecta and rare chondrodysplasias and genetic diseases of muscle
(b) Haematological abnormalities associated with the loss of stromal function, including myeloma and myelodysplastic syndrome and others.

According to a further embodiment of the invention, the therapeutic regimens of the fifth and sixth aspects of the invention may be adapted such that the fraction isolated in step (c) is treated to genetically modify the enriched MSCs prior to being introduced (or re-introduced) into the subject.

It is preferred that MSCs from the subject (i.e. autologous MSCs) are genetically modified and re-introduced into the subject. However it will be appreciated that some clinical situations may dictate that MSCs from a cadaver or donor source may be required. In fact such MSCs (from a cadaver or donor source) may comprise a desired genotype. The cells may therefore be introduced into the subject to correct one genetic abnormality (as contemplated above) and may undergo genetic modification to have a further genetic influence in the subject being treated. Accordingly, cells enriched according to the present invention may be used in the treatment of a number of conditions with genetic components, these include those contemplated under (1)-(9) and particularly (10) and (11) above.

Kits according to the fourth aspect of the invention may comprise any collagenase discussed above. It is preferred that the collagenase digests Collagen I. The collagenase may be derived from Clostridium histolyticum (e.g. as supplied by Stemcell Technolgies Inc, Vancouver, CA).

The kit may optionally contain suitable diluents and buffers for the collagenase (e.g. phosphate buffered saline (PBS)-optionally comprising a final concentration of 20% Fetal Bovine Serum) and the sample (this may also be PBS, preferably containing 2% Fetal Calf Serum, or another physiologically acceptable buffer such as DMEM.)

When step (b) is to involve further purification of the MSCs it is preferred that the kit further comprises reagents for putting this into effect. Accordingly the kit may comprise reagents and apparatus suitable for magnetic beads isolation of MSCs. Accordingly the kit may comprise: MACS columns, microbeads, MACS buffer (contains 0.1% Bovine serum albumin) as discussed herein. Alternatively the kit may comprise FACS reagents for further purification of the released MSCs (e.g. antibodies listed in Table 2 and buffers (e.g. PBS)).

The invention will be illustrated further by Examples and with reference to the following drawings, in which:

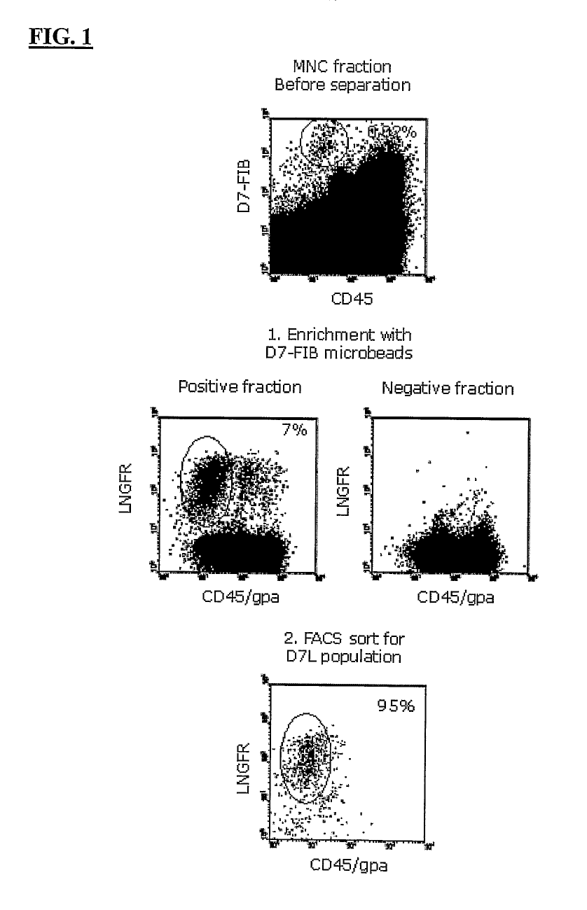

FIG. 1: illustrates the results of multiparameter flow cytometry of MSCs isolated from bone marrow (BM) as described in Example 1;

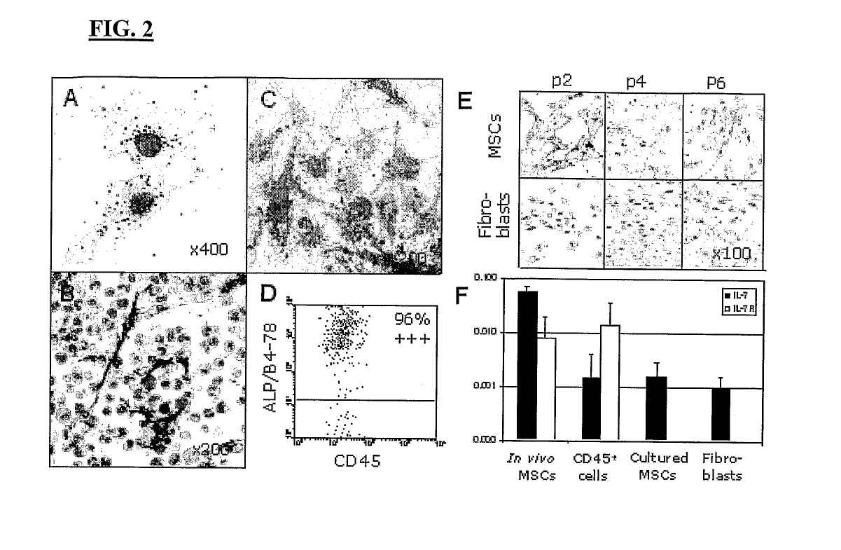

FIGS. 2: A, B, C and E illustrates histological analysis of MSCs in bone marrow (BM) and following expansion; D represent the results of flow cytometry analysis of expression of alkaline phosphatase by isolated MSCs; and F is bar chart illustrating change in levels of IL-7 and IL-7R expression by in vivo and cultured cells over time, as described in Example 1

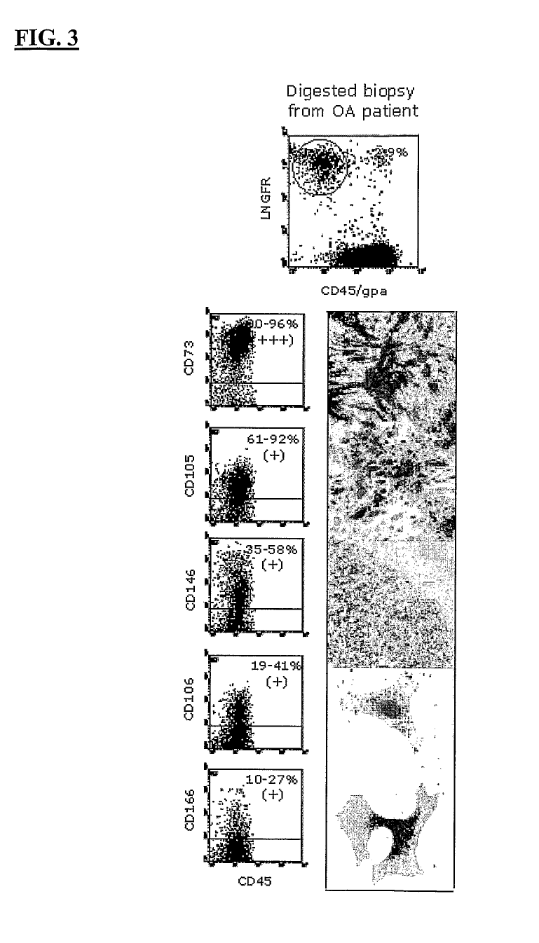

FIG. 3: comprises upper and lower panels. The upper panel of FIG. 3 illustrates FACS data indicating the high proportion of MSCs produced on enrichment of bone marrow samples by the methods of the present invention. The lower panel of FIG. 3 sets out flow cytometry data and photomicrographs illustrating that these BM MSCs enriched in accordance with the invention have the MSC phenotype and are able to give rise to a range of different cell lineages in keeping with their multipotent status.

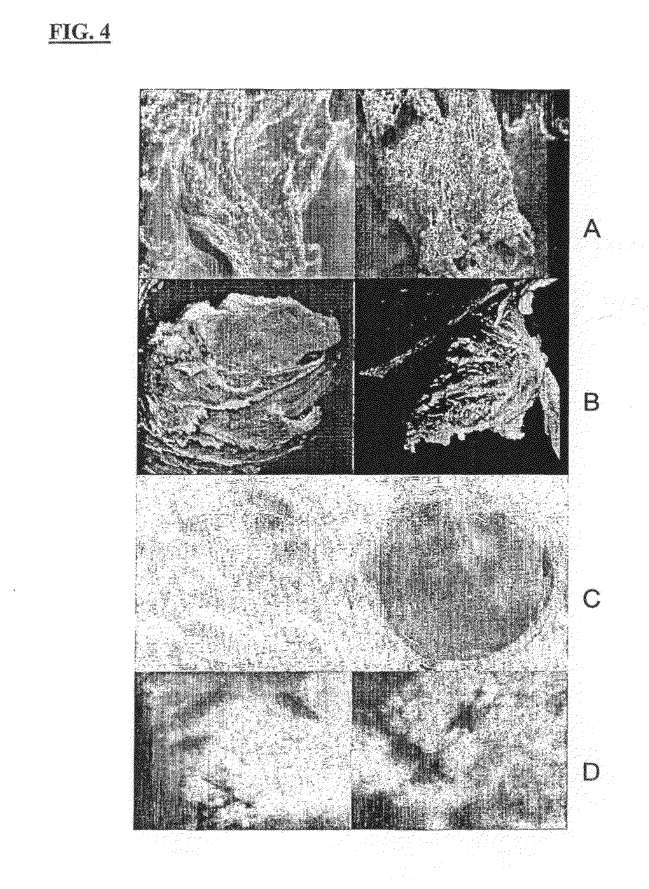

FIG. 4: further illustrates the usefulness of collagenase digestion procedures for the isolation of bone-lining cells/cell fractions with higher osteogenic capacity. A and B—scanning electron microscopy (A) and fluorescent confocal microscopy (B) images of BM cavity before (left) and after (right) collagenase digestion step, showing removal of most, but not all, bone-lining cells. The right hand panel of (B) illustrates a reduction in cells (as less DAPI stained blue cells on the right compared to the left on the colour image) on the bone surface (shown in green calcein staining on the colour image). (C) illustrates increased numbers of CFU-Osteoblast as the darker colour (osteogenic progenitors, Alizarin Red stain) in a second collagenase digest (right) compared to the first collagenase digest (left). Second digest removes residual bone-lining cells, and only cells that are left behind are osteocytes (D), which are trapped in bone itself and hence inaccessible to the enzyme (fussiness in some areas indicates that osteocytes are located on different planes).

FIG. 5: also comprises upper and lower panels. The upper panel of FIG. 5 illustrates FACS data indicating the surprisingly high proportion of MSCs produced on enrichment in accordance with the present invention of the floating fat fragment derived from bone marrow samples. The lower panel of FIG. 5 sets out flow cytometry data and photomicrographs illustrating that these MSCs enriched from the FFF in accordance with the invention have the MSC phenotype and are able to give rise to a range of different cell lineages indicating their multipotent status.

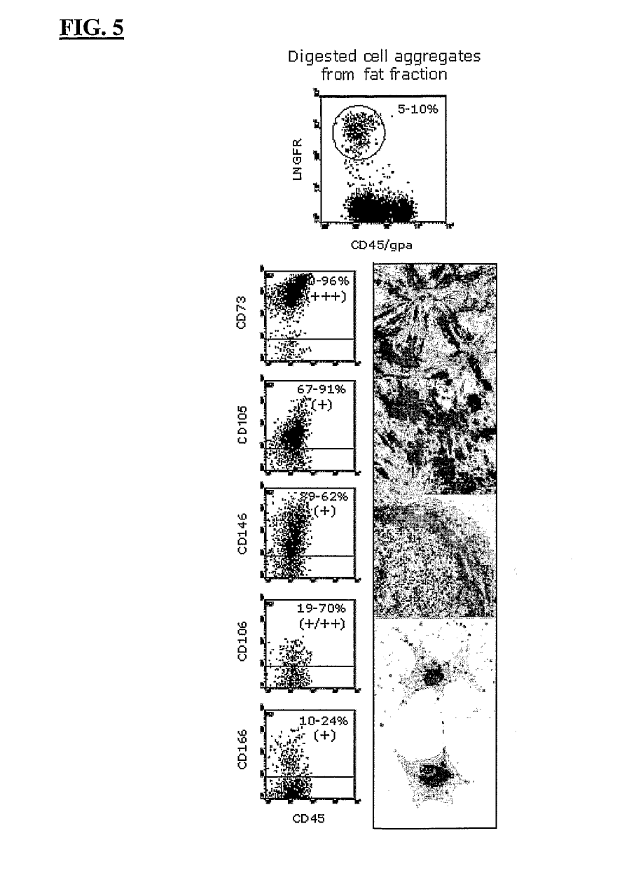

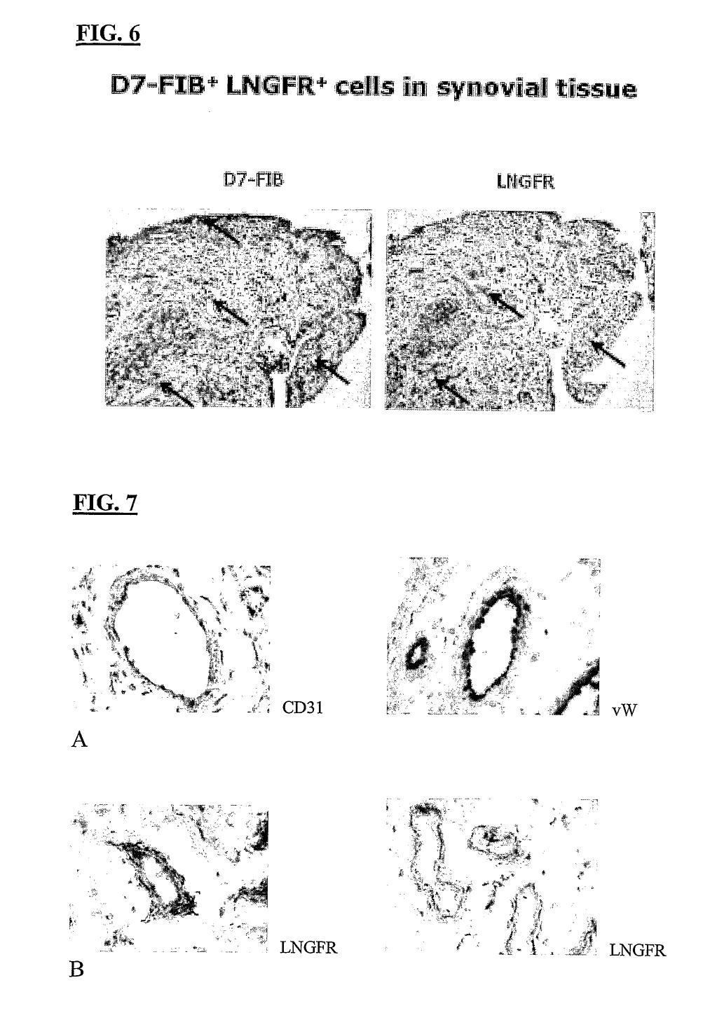

FIG. 6: represents photographs of histological sections illustrating that D7-FIB antibody stained all fibroblasts, whereas LNGFR stained only fibroblasts in perivascular areas as discussed in Example 2.

FIG. 7: represents photographs of histological sections contrasting the distribution of endothelial cells of the blood vessel lining and MSCs associated with blood vessels within the tissue samples discussed in Example 2.

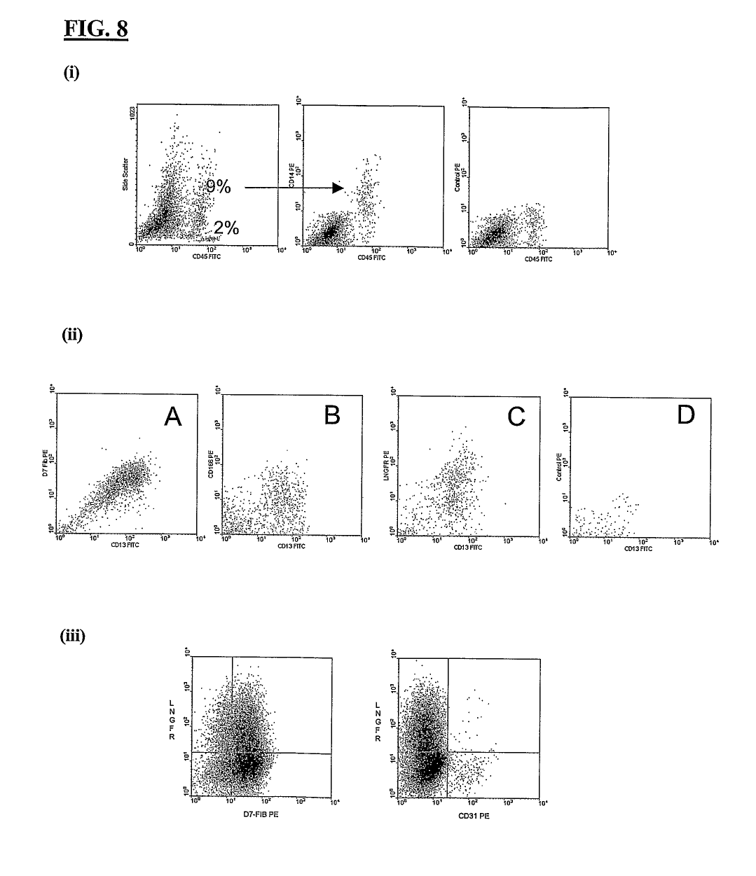

FIG. 8: illustrates a representative experiment in Example 2 that illustrates expression of LNGFR in MSCs enriched from synovium in Example 2. Panel (i) shoes a gout synovial tissue primary digest for which dead/dying cells were removed by PI gating. The majority of cells are non-hematopoietic with a small proportion of lymphocytes (2%) and larger number of monocytes/macrophages (MM) (9%) as identified based on their CD45/SSC profiles. MM have low CD14, consisted with expression on macrophages. Panel (ii) illustrates dual labelling with CD13 and demonstrates that all synovial tissue fibroblasts (CD31+), express D7-FIB, another fibroblast marker (A); CD31+cells uniformly express low levels of CD166 (ALCAM), a putative MSC marker (B); LNGFR, a marker of BM MSCs, is also expressed by synovial tissue fibroblasts (CD13+) and shows a spectrum of positivity, from very bright to less bright cells (C). (D) represents control staining. Panel (iii) illustrates dual labelling with D7-FIB and demonstrates that LNGFR, a marker of BM MSCs, is expressed on synovial tissue fibroblasts (D7-FIB+) and shows a spectrum of positivity, from very bright to less bright cells (left panel). LNGFR is not expressed on synovial tissue endothelial cells (CD31+), confirming immunohistochemistry data.

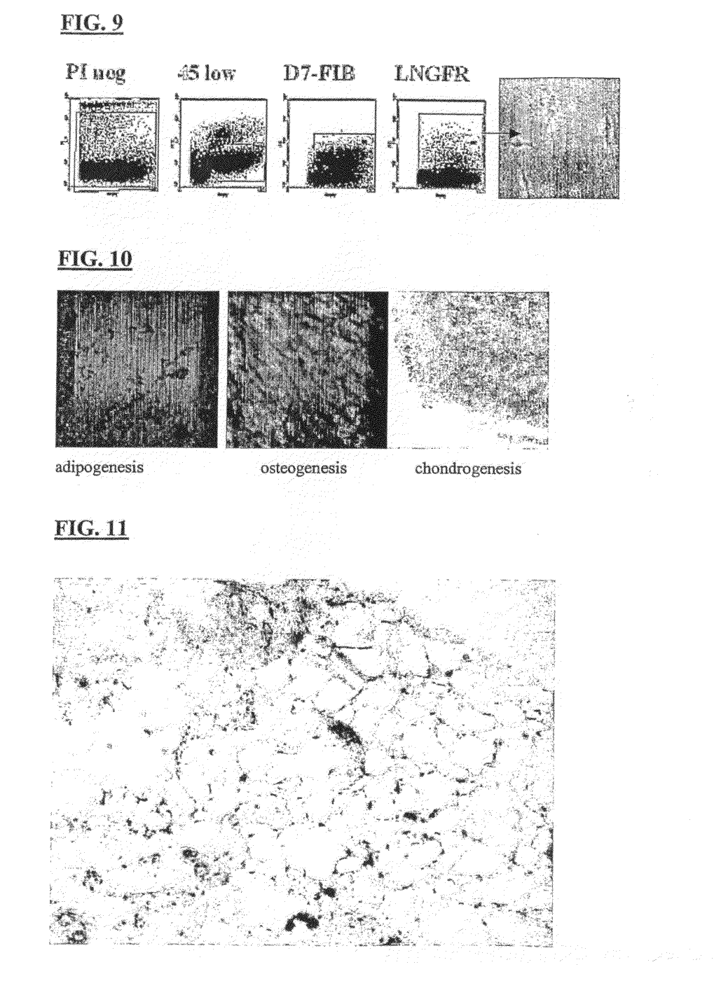

FIG. 9: illustrates the sorting strategy for isolating MSCs in Example 2 and also illustrates the morphology of cells produced by this enrichment strategy.

FIG. 10: represents photographs of cultures or histological sections across chondrogenic pellets illustrating the differentiation of MSCs isolated from Synovium and following in vitro culture as discussed in Example 2

FIG. 11: represents a photograph of a histological section illustrating that LNGFR⁺ cells in fat pads have perivascular topography as discussed in Example 3.

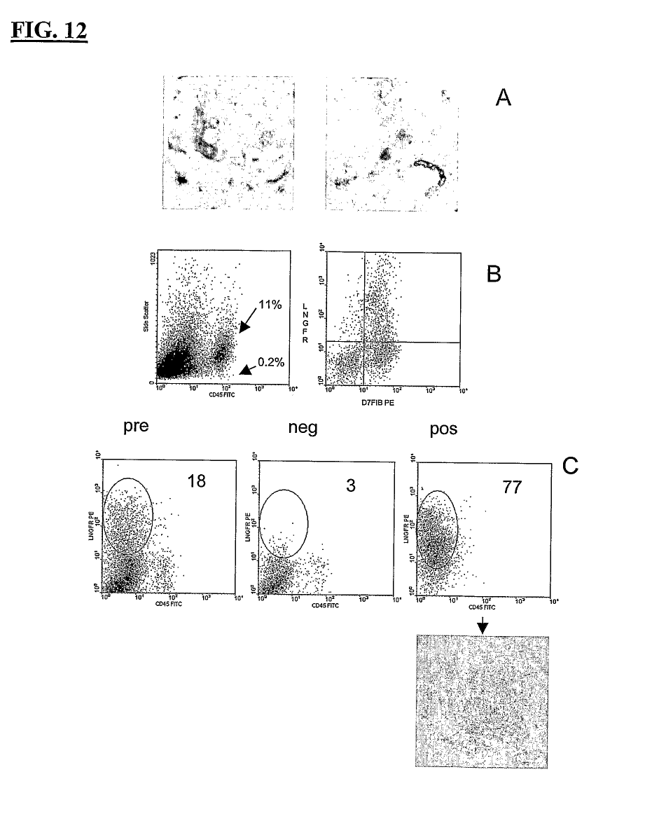

FIG. 12: also represents photographs of a histological section illustrating that LNGFR⁺ cells in fat pads have perivascular topography and further illustrates the phenotype and sorting strategy for fat pad MSCs as discussed in Example 3.

### EXAMPLE 1

### Mesenchymal Stem Cell enrichment from Bone Marrow Samples

### 1.1 Materials & Methods

### 1.1.1 Sample Collection

(i) BM aspirates (2-10 ml) were obtained from the posterior iliac crest of normal donors. BM mononuclear cells (MNCs) were collected following separation with Lymphoprep (Nycomed, Oslo, Norway). Cell aggregates concentrated in floating fat fraction (FFF) were manually dispersed by agitation and centrifuged. Uniocular adipocytes remained floating at the top whereas stromal cells were concentrated by in the pellet.

(ii) BM biopsy material was obtained from femoral heads removed during hip arthroplasty of consented patients with osteoarthritis (OA). Femoral heads were cut in half by a surgeon and delivered to laboratory in sterile containers with PBS. Specimens were collected using a 4-mm punch biopsy. Alternatively, femoral heads were homogenized using Bone Mill (De Puy or other manufacturers) to obtain even smaller fragments to undergo enzymatic digestion.

### 1.1.2 Collagenase Digestion

0.1 g punch biopsy samples were digested with 0.5 ml of 0.25% collagenase (Stem Cell Technologies, Vancouver, Canada) for 3-4 hours. This corresponded to approximately 250-375 Units of enzyme. The inventors found this optimal for cell recovery and viability.

### 1.1.3 Handling of MSC Fraction

At the end of the preferred incubation time, collagenase solution was diluted with an over 20-fold excess of PBS (v/v) and a syringe/needle was used to mix the released cells with PBS and to enforce remaining cells to egress from the biopsy into the solution. Immediately after, PBS/cell mixture was sieved trough 70 micron cell strainer and transferred into another centrifuge tube for centrifugation (5 minutes at 1800 rpm). Collagenase/PBS solution was then discarded and the cell pellet was re-suspended in media appropriate for cell counting and subsequent manipulations (magnetic and FACS separation), usually DMEM with 2% FCS.

From some samples, CD45^{low}D7-FIB⁺LNGFR⁺ cells were processed for flow cytometry, purified, expanded and differentiated, as previously described. (Jones et al. Arthritis Rheum 2002:46: 3349-60).

### 1.2 Results

1.2.1 Confirmation that Rare Mesenchymal Stem Cells (MSC) in the Bone Marrow (BM) aspirates have a D7-FIB⁺CD45^{low}LNGFR⁺ phenotype

The inventors have established that in BM aspirates all MSC activity was confined to a rare population of CD45^{low}D7-FIB⁺LNGFR⁺ cells. They further investigated the frequency and clonogenicity of this population. For the same donors, the average frequency of CD45^{low}D7-FIB⁺LNGFR⁺ cells in BM (mononuclear cell) MNC fraction, measured by 4-colour cytometry, was 0.039±0.037% (FIG. 1, top) and was comparable in magnitude to the average CFU-F frequency of 0.007±0.006% (n=30) occurring in the same samples.

Furthermore, the inventors found that the frequency of this rare cell population was increased ˜100-fold (up to 7±6%, n=38) following pre-enrichment with D7-FIB microbeads (FIG. 1, middle panel). This pre-enrichment step is a positive selection with microbeads directly-conjugated to D7-FIB (Anti-Fibroblast Microbeads, Miltenyi Biotec). Briefly 10⁸ BM MNCs are incubated with 100 μl microbeads for 15 min at RT and then washed at 1800 rpm×5 min to remove unbound microbeads. Then cells are applied into a MiniMacs column (placed in a magnet) and unbound fraction is collected in 6 ml of MACS buffer (PBS with 0.5% BSA). Subsequently, the column is removed from a magnet and the bound fraction (containing MSCs) is released in 0.5-1 ml of MACS buffer. This is a standard protocol of MACS separation.

Following FACS sorting to select the CD45^{low}D7-FIB⁺LNGFR⁺ population, the purity of the CD45^{low}D7-FIB⁺LNGFR⁺ cell population was increased to over 95% (FIG. 1, bottom panel). Of this enriched population, one in 6 cells (17±4%, n=6) formed defined colonies of 30 or more cells in a standard CFU-F assay (Castro-Malaspina et al. (1980) Blood 56 p289-301).

No colonies were grown from matching CD45⁺ cells. These data confirmed that all clonogenic MSCs in BM aspirates were contained within the rare CD45^{low}D7-FIB⁺LNGFR⁺ population.

### 1.2.2 Stromal Features of In Vivo BM MSCs

The inventors noted that MSCs purified from BM aspirates displayed a stromal morphology (star-shaped with numerous projections), reminiscent of BM adventitial reticular cells (ARCs), and that small fat droplets were found in their cytoplasm (FIG. 2A). On BM biopsies, cells with ARC morphology were positive for the CD10 marker, which was expressed on in vivo MSCs (FIG. 2B). Moreover, purified in vivo BM MSCs were uniformly positive for alkaline phosphatase (ALP), which is the main feature of ARCs (FIG. 2C). Staining with an antibody against ALP (B4-78 clone, against ALP bone/liver isoform) demonstrated a uniform expression on all in vivo MSCs (FIG. 2D). ALP expression gradually disappeared following MSC culture expansion (FIG. 2E). Expression of IL-7, a cytokine crucial for stromal support of lymphopoiesis and known to be produced by ARCs, also declined following expansion (FIG. 2F). Cultured skin fibroblasts were always negative for ALP and expressed minimal IL-7 (FIGS. 2, E and F).

The inventors were surprised to realise, when these data are taken together, that BM MSCs in vivo had many features consistent with ARCs and these characteristics were lost following in vitro culture. This suggested two things. First, culturing of MSCs may be undesirable because it leads to a phenotypic change. Second, a skilled person would not have appreciated that MSCs in vivo have ARC like properties. ARCs form an interconnected network of cells and may be freed with enzymatic treatment. Accordingly the inventors appreciated that it would also be possible to isolate MSCs using enzymatic digestion. The inventors believe that MSCs activity resides in ARCs and maybe in other previously overlooked cells such as bone-lining cells, pericytes and preadipocytes. All of these are not possible to aspirate and that's why enzymatic digestion is needed.

1.2.3 Enzymatic Release of In Vivo MSCs from BM Stromal Cell Aggregates

Having realised that MSCs may have ARC-like properties, the inventors investigated whether or not enzymatic digestion of a tissue would liberate MSCs. They tested this idea on material obtained from femoral heads removed during hip arthroplasty of patients with osteoarthritis (OA).

MSCs represent a very rare population in single cell suspensions from BM aspirates. An average cell yield of highly pure MSCs (CD45^{low}D7-FIB⁺LNGFR⁺) from 10⁸ aspirated BM MNCs was 11,000 cells (range 4,000-63,000, n=14), equivalent to ˜2,000 MSCs per millilitre of aspirated marrow. This yield is too low to be clinically useful and requires in vitro expansion to generate useful numbers of cells. Furthermore, as indicated above, the inventors believe that an expansion step may lead to a change in MSC phenotype.

Having realised that in vivo BM MSCs share many of the same characteristics as ARC cells, the inventors next tested whether enzymatic methods of extraction, employing the methods described in 1.1 (based on those able to break-up the ARC/matrix network), would be suitable to liberate increased numbers of MSCs from solid tissue biopsies, as opposed to conventional bone marrow aspirate sources.

Three-hour, or three to four-hour, collagenase treatment of biopsies liberated CD45^{low}″D7-FIB⁺LNGFR⁺ cells, which were morphologically and phenotypically similar to MSCs purified from aspirates but, surprisingly, the proportion of those cells in populations liberated from biopsy samples was ˜100-fold higher than the levels found in aspirates (5±3% live cells, n=8, as opposed to 0.039-10.037% live cells, n=30, in BM aspirates). This increase is indicated in the FACS results shown in the top panel of FIG. 3.

Furthermore, the inventors surprisingly found that, despite originating from a diseased microenvironment, CD45^{low}D7-FIB⁺LNGFR⁺ cells purified from digested biopsies in accordance with the method of the first aspect of the invention retained normal MSC function in respect of proliferation and differentiation, although the level of chondrogenesis which was diminished (1±1 μg of sulphated glycosaminoglycan (GAG)/pellet compared to 3±2 μg, produced by normal BM controls, n=4). This finding was consistent with known features of MSCs derived from BM of OA patients. When trabecular bone biopsies from normal individuals were used, normal levels of MSC chondrogenesis were obtained (data not shown).

The results of this study are shown in the lower panel of FIG. 3, which illustrates photomicrogaphs and FACS analysis of MSCs enriched according to the method of the first aspect of the invention. The FACS results shown in the left hand figures of this panel illustrate expression on gated CD45^{low}D7-FIB⁺LNGFR⁺ cells (i.e. fresh MSCs) of markers illustrative of their MSC nature. The photomicrographs shown in the right hand figures of this panel illustrate that CD45^{low}D7-FIB⁺LNGFR⁺ cells obtained by collagenase digestion are able to differentiate to three mesenchymal lineages.

Yields of highly enriched MSCs (CD45^{low}D7-FIB⁺LNGFR⁺) sorted from digested biopsies were on average 50-fold (and in some cases over 100-fold) higher than those normally obtained from aspirates (˜550,000 cells per 10⁸ total cells, range 80,000-800,000, n=6, as compared to 11,000 cells, range 4,000-63,000, n=14, from 10⁸ aspirated BM MNCs). On average, 0.1 g biopsy yielded approximately 150,000 CD45^{low}D7-FIB⁺LNGFR⁺ cells equivalent to approximately 25,000 CFU-Fs (n=7). The proportion of CFU-Fs in a released cell fraction directly correlated with the frequency of CD45^{low}D7-FIB⁺LNGFR⁺ cells (R=0.89, n=7). Thus the inventors were able to obtain approximately 10,000 MSCS or 25,000 or 150,000 MSCs (depending on the method of enumeration, CFU-F assay or flow cytometry, respectively) from a single 0.1 cm³ (0.1 g in weight) biopsy. The inventors were surprised to realise that this yield was sufficiently high to allow direct clinical use of the MSCs derived by this method. These yields would be equivalent to using between 150 and 350 millilitres of a BM aspirate.

Indeed, the inventors found that the frequency of CD45^{low}D7-FIB⁺LNGFR⁺ cells released by collagenase digestion in accordance with the method of the first aspect of the invention (2-9%, n=8) was so high, that pre-enrichment with microbeads was no longer required in order to enable their detection, isolation or use as therapy. On the other hand, if the same MSC dose from conventional BM aspirate were used for the same purpose (i.e. 150-350 millilitres), MSCs would have to be considerably concentrated to ensure similar levels of purity. This illustrates how advantageous the inventor's methods are because the invention allows a clinician to quickly utilise MSCs taken from a sample without the need for complicated and time consuming enrichment step or expansion in culture. Moreover, the inventors have established that one femoral head from a single patient can be processed according to the methods of the invention and provide enough MSCs for multiple treatments which would not be possible if BM aspirates were used.

1.2.3 Enzymatic Release of Highly Osteogenic MSCs Using Double Digestion with Collagenase

For bone repair applications, the use of MSCs with predominant osteogenic capacity is desirable. Having realised that MSCs activity can reside in bone-lining cells, the inventors were able to show that additional enzymatic methods of extraction, over and above 3-4 hour collagenase digestion step, as described in 1.1, were suitable to liberate MSCs with increased osteogenic capacity. As seen on FIGS. 4, A and B, the inventors were able to demonstrate that collagenase digestion step, employing the methods described in 1.1, removed most, but not all bone-lining cells (an average of approximately 2% of total DNA was left on bone, n=3). When a second 1-hour collagenase digestion step was performed on biopsy fragments following the collection of the first digest, the proportion of cells left on bone decreased by nearly 5-fold and the total yield of recovered cells increased by an average of 5% (n=3). Although clonogenicity of the second digest was similar to that of the first digest (13 CFU-Fs/5,000 released cells compared to 12 CFU-Fs/5,000 released cells, respectively, n=3), its osteogenic potential was often twice as high (170 μg Ca⁺⁺/5,000 released cells compared to 90 μg Ca⁺⁺/5,000 released cells, respectively) (FIG. 4C). The inventors were also able to demonstrate that the second collagenase digestion step could be substituted with 1-hour treatment with 0.5 ml solution of hyaluronidase (Sigma) diluted in PBS to a final concentration of 50 U/ml. Similar to collagenase digestion, treatment with hyaluronidase is performed at 37° C. on a cell shaker. This treatment also liberated bone-lining cells which contributed to extra 5% of total cells released (n=3). The MSC marker phenotype of CD45^{low}D7-FIB⁺LNGFR⁺ cells in second digests, obtained either using collagenase or hyaluronidase, was similar to that of the first digests (CD73⁺⁺CD105⁺⁺CD90⁺⁺⁺CD146⁺⁺, n=2). Following this double digestion procedure the only residual cells that remained in situ were located inside the bone (osteocytes) (FIG. 4D).

It will therefore be appreciated that a preferred method of processing bone, samples, for bone repair applications is to conduct a second collagenase treatment or alternatively a hyaluronidase treatment as described above.

1.2.3 Enzymatic Release of In Vivo MSCs from Floating Fat Aggregates

Normal BM contains buoyant fat droplets, termed the floating fat fraction (FFF), which is discarded, by density gradient centrifugation, during the preparation of BM MNCs as a prelude to MSC enrichment. The FFF is increased in aged individuals (as their marrow progressively becomes fattier) or people with some diseases (such as rheumatoid arthritis or osteoporosis).

The inventors have surprisingly found that the method of the invention can be applied to aspirates to increase MSCs yields. This requires the collection of the FFF. It is then collagenase-treated according to the invention to separate stromal cells/ARCs/MSCs from mature adipocytes.

The inventors analysed floating fat fraction (FFF) aggregates in aspirates collected from normal BM using conventional prior art techniques. In studies using the same donors the proportion of cells with a CD45^{low}D7-FIB⁺LNGFR⁺ phenotype in dispersed FFF was 0.3±0.1%, hence ˜10-fold higher, than the proportion present in the corresponding MNC fraction (0.03±0.006, n=5). Thus the inventors surprisingly found that the FFF, which would normally be discarded in accordance with prior art techniques, represents a valuable source of MSCs.

Furthermore, the inventors found that when FFF cells were digested with collagenase, the proportion of CD45^{low}D7-FIB⁺LNGFR⁺ cells yielded was increased further and reached 7±5% of total live cells (n=5) (FIG. 5, top panel). This yield was ˜200-fold higher than in the MNC fraction that is used as a source of MSCs according to prior art techniques. CD45^{low}D7-FIB⁺LNGFR⁺ cells released from the FFF had a similar extended phenotype compared to their MNC counterparts, and were multipotential following expansion (as shown in the bottom panel of FIG. 5 which illustrates FACS results and morphology studies investigating the same cell lineages described above). The CD45^{low}D7-FIB⁺LNGFR⁺ cells enriched from the FFF were ALP-positive and had small fat droplets in their cytoplasm. This indicates the cells have a “pre-adipocyte” state in vivo. Their expression of alkaline phosphatase in vivo suggests their “pre-osteoblast” state. Taken together, this suggests multipotentiality inherent to individual single cells.

Taken as a whole, these data showed that the majority of MSCs present in the BM are not found in the form of single cells (of the type which may be expected to be collected using aspirates as suggested by the prior art). Instead, it was surprisingly found that the majority of BM MSCs were integrated into the stromal cell network, forming aggregates with neighbouring cells, including buoyant fat cells, and collagenous matrix.

It will be recognised in the light of this surprising finding that the method of the first aspect of the invention (utilising collagenase digest to free MSCs from the network of stromal cells and matrix) may be used to isolate and enrich MSCs from a far greater starting population than may otherwise be achieved using prior art techniques. Thus the method of the first aspect of the invention provides a new and inventive method by which increased enrichment of MSCs may be achieved.

### EXAMPLE 2

### Mesenchymal Stem Cell Enrichment from Synovium

### 2.1 Materials & Methods

### 2.1.1 Sample Collection

Samples from eight synovial tissue sources were investigated: from one normal donor, from four patients with RA, one patient with OA, one patient with gout and one patient with seronegative arthritis.

Synovial biopsies were taken during diagnostic arthroscopy. Small tissue biopsy (average 44 mg) was placed in a 15-ml centrifuge tube with 2 ml of media (normally DMEM/2% FCS) to be transported to the laboratory.

### 2.1.2 Collagenase Digestion

Upon arrival into the laboratory, media was removed and biopsy was placed in 0.25% collagenase solution and digested for 3 or 4 hours under constant rotation. When the sample is completely digested, collagenase solution becomes cloudy and cell biopsy disintegrates and disappears completely. After incubation, 10 ml PBS was added and syringe/needle were used to break any possible remaining tissue clumps. Immediately after, PBS/cell mixture was sieved trough 70 micron cell strainer and transferred into another centrifuge tube for centrifugation (5 minutes at 1800 rpm). Collagenase/PBS solution was then discarded and the cell pellet was re-suspended in media appropriate for cell counting and subsequent manipulations (magnetic and FACS separation), usually DMEM with 2% FCS.

### 2.1.3 Cell Sorting Strategy

Cells liberated from synovial samples using the collagenase digestion technique described above were enriched for CD45^{low}D7-FIB⁺LNGFR⁺ cells using the protocol shown schematically in FIG. 9. Briefly, dead cells were discarded on the basis of high propidium iodide (PI) staining, and the remaining live cells sequentially sorted to select those having low CD45 expression but high expression of LNGFR.

The fibroblastic morphology of sorted cells yielded by this strategy is illustrated in the last image of FIG. 9. These sorted CD45^{low}D7-FIB⁺LNGFR⁺ cells were subsequently able to generate growing cell monolayers in culture, consistent with their high proliferative nature.

### 2.2 Results

### 2.2.1. Immunochemistry and Flow Cytometry Analysis

Mesenchymal stem cells have been reported in many other tissues but their in vivo topography and phenotypes have not previously been known. The inventors investigated the topography of CD45^{low}′D7-FIB⁺LNGFR⁺ cells in solid tissues including synovium and joint fat pads. In synovium, D7-FIB antibody stained all fibroblasts, whereas LNGFR stained only fibroblasts in perivascular areas (FIG. 6).

Results illustrating the pericyte-like distribution of CD45^{low}D7-FIB⁺LNGFR⁺ cells are illustrated in FIG. 7. CD45^{low}D7-FIB⁺LNGFR⁺ cells were distinct from endothelial cells (known to be CD31⁺ and von Willebrand factor⁺), which formed a layer one cell in depth inside the lumen of blood vessels (immunolabeling shown in FIG. 7A).

In contrast, CD45^{low}D7-FIB⁺LNGFR⁺ cells formed the blood-vessel support as several layers of positive cells extending into tissues, consistent with a notion that MSCs in solid tissues are pericytes (FIG. 7B).

2.2.2 Enzymatic Release of MSCs from Synovium

MSCs were liberated from synovium using the methods outlined in 2.1 above.

Enzymatic digestion of synovium in accordance with the method of the first aspect of the invention yielded a large proportion of CD45^{low}D7-FIB⁺LNGFR⁺ cells, as shown in FIG. 8. This enrichment of CD45^{low}D7-FIB⁺LNGFR⁺ cells was found to be irrespective of the health status of the sample from which the cells were isolated (e.g. the tissue sample from gout in panel further illustrating the suitability of the method of the first aspect of the invention for the therapeutic isolation of autologous MSCs, even from patients suffering from disease.

CD45^{low}D7-FIB⁺LNGFR⁺ cells were enriched from diverse synovial samples (one from a normal donor, four from patients with RA, one from a patient with OA, one from a patient with gout and one from a patient with seronegative arthritis). Independent of the nature of the disease, CD45^{low}LNGFR⁺ cells were present in all synovial digests, at an average proportion of 15±9% and in other data sets of 16±10%.

In keeping with the immunohistochemistry data showing that all fibroblasts label positively for D7-FIB, CD45^{low}D7-FIB⁺ cells were present in an increased proportion in the synovial digests (an average proportion of 76±20% and in another data set of 50±12%).

A representative experiment demonstrating the expression of LNGFR and D7-FIB on collagenase digests of synovial tissue is shown on FIG. 8, panels (ii) and Panel (iii) in particular shows that CD45^{low}LNGFR⁺ cells (synovial pericytes) constitute a fraction of CD45^{low}D7-FIB⁺ cells (synovial fibroblasts), consistent with immunohistochemistry data. Commonality in the phenotype suggests their common mesenchymal lineage nature and a possibility of the MSC presence in the cells with both phenotypes.

2.2.3. Functional Evidence that Sorted Cd45^{low}LNGFR⁺ Cells Contain MSCs

Cell monolayers (passage 4) were generated from sorted CD45^{low}D7-FIB⁺LNGFR⁺ cells from synovial tissue (i.e. from synovial pericytes) enriched in accordance with the method of the first aspect of the invention. These cell monolayers were subjected to functional in vitro assays of osteogenic, adipogenic and chondrogenic differentiation (n=1) as described below.

i) Osteogenic differentiation was induced by 100 nM dexamethasone, 0.05 mM L-ascorbic acid-2-phosphate, and 10 mM (3-glycerophosphate (all from Sigma). Alkaline phosphatase (AP) activity and matrix mineralization were detected using the Sigma kit 82 and 1% Alizarin Red (Sigma), respectively. Ca⁺⁺ deposition was measured using Sigma kit 85.

ii) Adipogenic differentiation was induced in DMEM/10% FCS, supplemented with 0.5 mM isobutilmethylxantine (Sigma), 60 μM indomethacine (ICN, Basingstoke, UK), and 0.5 mM hydrocortisone (Sigma). Accumulation of lipid vacuoles was visualized with 0.5% Oil Red, as previously described.

iii) For chondrogenic differentiation 2.5×10⁵ cells were placed in serum-free media consisting of high-glucose DMEM (Gibco), 100 mg/ml sodium pyruvate, 40 mg/ml proline, 50 μg/ml L-ascorbic acid-2-phosphate, 1 mg/ml BSA, 1×ITS+, 100 nM dexamethasone (all from Sigma) and 10 ng/ml TGF-β3 (R&D Systems). Media was changed every other day. Pellets were harvested at week 3 and frozen sections (5-μm thick) were prepared. Sulfated GAG was visualized with 1% Toluidine Blue (Sigma). sGAG was measured using alcian blue-binding assay (IDS).

Following three-week differentiation, cultures derived from the CD45^{low}D7-FIB⁺LNGFR⁺ cells liberated from synovial tissue in accordance with the invention successfully differentiated towards adipogenic, osteogenic and chondrogenic phenotype. The results of this study are shown in FIG. 9, which sets out photomicrographs illustrating the phenotypes achieved by the cultured cells.

Multipotentiality of cells derived from sorted synovial CD45^{low}D7-FIB⁺LNGFR⁺ cells was compared to that of cultures derived from the BM (n=5) or synovium (n=5), in which cultures were generated by standard plastic-adherence method. The proportions of generated adipocytes were similar (30% versus 33±15% and 22±15%, respectively) and the amounts of calcium produced (indicative of osteogenesis) were within the same range (100 μg/dish versus 80±37 μg and 121±47 μg, respectively). Chondrogenesis, in particular, was very strong and the amount of produced proteoglycans (GAG) was above control cultures (9 μg/pellet versus 2.6±1.9 μg μg/pellet and 2.2±1.4, respectively).

The data set was expanded such that a comparison was made with n=15 for synovium. Similar results were obtained and in this case the amount of produced proteoglycans (GAG) was above control cultures (9 μg/pellet versus 2.6±1.9 μg μg/pellet and 1.8±1.7, respectively). Overall, CD45^{low}D7-FIB⁺LNGFR⁺ fraction contained over two thirds of the total digest's chondrogenic activity, with only ⅓ of it being left in the residual fraction. These data showed that synovial CD45^{low}D7-FIB⁺LNGFR⁺ cells (synovial pericytes) contained MSC activity, comparable to control cultures, and were specifically enriched for chondrogenic activity.

2.2.4 Confirmation at the Single-Cell Level of MSC Presence within Synovial Cd45^{low}D7-FIB⁺LNGFR⁺ population enriched in accordance with the invention.

The inventors investigated the clonogenic capacity of individual cells liberated from synovial tissues in accordance with the present invention (n=3 donors, all with RA).

CD45^{low}D7-FIB⁺ cells (synovial fibroblasts) were purified by FACS sorting using the protocol shown schematically in FIG. 9. On average 10% of adherent cells sorted by this protocol were able to undergo 12-13 cell divisions, however only 25% of these (or 2.5% of total adhered CD45^{low}D7-FIB⁺ cells) were highly proliferative (capable of undergoing 20-22 cell divisions).

In addition, cells liberated by collagenase digest of one synovial tissue (RA), were sorted to select the CD45^{low}D7-FIB⁺LNGFR⁺ population (synovial pericytes). In this experiment 4% of total adhered CD45^{low}D7-FIB⁺LNGFR⁺ cells were highly proliferative and clonogenic (capable of undergoing 20-22 cell divisions). All three single MSC-derived clones were tripotential (FIG. 10).

These experiments showed that highly clonogenic and multipotential MSCs were present within populations of both synovial fibroblasts and synovial pericytes released from synovial tissues by collagenase digest in accordance with the present invention and that they were numerically enriched in the pericyte fraction (CD45^{low}D7-FIB⁺LNGFR⁺ cells). Furthermore, these results illustrate that enriched synovial perivascular CD45^{low}D7-FIB⁺LNGFR⁺ cells are a particularly good source of chondroprogenitors.

The cell yield of synovial pericytes that could be obtained from one small 44 mg biopsy was on average 400,000 cells (n=8). This amount is sufficient for the treatment of a small focal cartilage defect (cell density currently used for standard autologous chondrocyte implantation (ACI) procedures is 10⁶ cells/cm²). If synovial fibroblasts or total synovial tissue digests are used, a much larger chondral defects can be treated (up to 10 cm²) using one small biopsy. Synovial tissue has a high capacity for regeneration and the collection of several biopsies or larger parts of synovium would enable the treatment of very large chondral defects as seen in OA and RA. High proliferative and clonogenic activity of synovial pericytes suggests that much smaller cell seeding densities (e.g. initial cell numbers) may be used, compared to conventional ACI standards.

### EXAMPLE 3

### MSCs are Present in Infrapatella Fat Pads

MSCs (indicated by LNGFR⁺ immunolabeling) in joint fat pads had perivascular topography similar to the topography of such cells in synovium, as illustrated in FIGS. 11 and 12, A.

Infrapatella fat pads (n=4 in preliminary experiments and the expanded to n=6, all patients with OA) were subjected to collagenase digest in accordance with the invention. Flow cytometry data confirmed that fat pad pericytes (CD45^{low}LNGFR⁺ cells) contributed a fraction of fat pad fibroblasts (CD45^{low}D7-FIB⁺ cells) (FIG. 12, B). Flow cytometry indicated that the average proportion of CD45^{low}′LNGFR⁺ cells was 20±12% in the preliminary experiments (n=4) and even better when n=6 (37±9).

The proportion of CD45^{low}D7-FIB⁺ cells (fibroblasts) in such digests was also investigated by flow cytometry, and the results indicated that (in keeping with the data from synovial studies), these cells constituted a larger proportion of the digest (60±12% (n=4) and 51±17% (n=6)). In comparison to synovial digests that we analysed, the proportion of fibroblasts in the fat pads was similar (50 versus 51%, respectively), however the proportion of pericytes (CD45^{low}LNGFR⁺ cells) was twice as high (37 versus 16%, respectively). This suggested that in terms of cell yields, fat pad may be a particularly useful tissue source for the selection of MSCs.

To purify CD45^{low}D7-FIB⁺LNGFR⁺ cells (pericytes) from fat pad digests, the inventors used a microbead-based technology with MACSelect Anti-LNGFR microbeads (FIG. 12, C). The MACS elect Anti-LNGFR microbeads are available from Miltenyi Biotec. Selection protocol is exactly the same as one described for D7-FIB microbeads (see above).

Cell purifies after this magnetic sorting procedure (n=4) were lower than to those obtained by direct FACS sorting (˜60-70% purity versus 95%) (FIG. 20, C). However the inventors believe this may be due to limitations of the method of assessing purities rather than actual purities themselves. Despite these limitations, clonogenicity of fat pad-derived CD45^{low}LNGFR⁺ pericytes from collagenase digests was similar to that of synovium-derived CD45^{low}LNGFR⁺ cells (4% of total adhered CD45^{low}LNGFR⁺ cells). A colony derived from sorted fat pad pericytes is shown on FIG. 12, C.

Sorted fat pad-derived CD45^{low}LNGFR⁺ pericytes had some multipotentiality even before culture expansion. This was possible to assess because the numbers of released/sorted cells were sufficient for in vitro testing in differentiation assays.

The results of these assays are shown in Table 3, which compares the properties of:
i) CD45^{low}LNGFR⁺ cells produced by collagenase digest; and
ii) CD45^{low}LNGFR⁻ cells produced by collagenase digest;
iii) control fat pad-derived plastic-adherent cells following expansion in culture (standard method).

The cells from (iii) above were obtained using standard expansion protocols. Briefly: fat pads were digested according to a standard protocol. Between 5×10⁵ and 10⁶ cells were seeded into a small 25 cm² flask and cultured until reached confluence. Confluent cells were trypsinized and split into two further flasks until confluence. Expanded cells (passage 1) were used to test multipotentiality as described above.

TABLE 3

| Cells | Donor | #1 | #2 | #3 | #4 | Average |
|---|---|---|---|---|---|---|
| CD45^{low}LNGFR⁺ | Ca++ | 5 | 9 | ND | 2 | 4 |
| cells | GAG | 0.5 | 0 | 9 | 0 | 2.4 |
| | Adipocytes | ~50% | ~50% | ~50% | ~50% | 50% |
| CD45^{low}LNGFR⁻ | Ca++ | 2 | 2 | 0.5 | 0.5 | 1.2 |
| cells | GAG | 0 | 0 | 4 | 0 | 1 |
| | Adipocytes | ~50% | ~50% | ~50% | ~50% | 50% |
| Expanded fat pad | Ca++ | ND | ND | 18 | 37 | 27 |
| cells | GAG | ND | ND | 13 | 6 | 9 |
| | Adipocytes | ND | ND | 70% | 64% | 67% |

As can be seen in Table 3, fat pad-derived CD45^{low}LNGFR⁺ cells were more chondrogenic than CD45^{low}LNGFR⁻ cells. On average, control cultured fat pad-derived MSCs produced higher chondrogenicity that fresh CD45^{low}LNGFR⁺ cells (9±7 versus 2.4±4.4, n=6 and n=4, respectively). It will be appreciated by those skilled in the art that differentiated cells derived in a patient from therapeutically administered MSCs enriched in accordance with the present invention will provide the increased and advantageous chondrogenic properties identified herein.

The cell yield of fat pad pericytes that could be obtained from one small 30 mg biopsy was in average 700,000 cells (n=2). This amount is sufficient for the treatment of a small focal cartilage defect (cell density currently used for standard autologous chondrocyte implantation (ACI) procedures is 10⁶ cells/cm). The cell yield of fat pad pericytes that could be obtained from one gram of tissue was in average 23×10⁶ cells (n=2). The cell yield of fat pad pericytes that could be obtained from the whole fat pad was in average 500×10⁶ cells (n=2). This would enable the treatment of very large chondral defects as seen in OA and RA. This will enable the use of a fat pad in allogeneic settings, providing chondrogenic fractions for numerous treatments.

Altogether these results demonstrated that CD45^{low}LNGFR⁺ cells resident in joint fat pads were:
i) perivascular,
ii) clonogenic; and
iii) multipotential.

These properties are all consistent with the identification of these cells as comprising mesenchymal stem cells.

Using the enrichment method of the invention it was possible to purify these cells in sufficient numbers (above 10⁶ cells) to test their multipotentiality directly, without resorting to in vitro culture expansion. The use of the infrapatellar fat pad a source of cells for enrichment by this method is particularly preferred due to the large size of this tissue and the relatively high proportion of CD45^{low}LNGFR⁺ cells present in the fat pad. These properties make an infrapatella fat pad an excellent source for obtaining unmanipulated MSCs utilising the methods of the first aspect of the invention. Such enriched cells are particularly suitable for tissue engineering applications.

In summary, these data indicate:
(1) For the first time freshly sorted cell populations were compared for multipotentiality with expanded cells and shown to have an MSC activity;
(2) CD45^{low}LNGFR⁺ cells were more chondrogenic and osteogenic than CD45^{low}LNGFR⁻ cells (accordingly CD45^{low}LNGFR⁺ represented a preferred phenotype for selection in step (b) of the first aspect of the invention; and
(3) Adipogenic activity of fresh cells was similar to expanded cells, hence one can simply collect and digest fat from one part of the body and inject/implant cells in the other part. Expanding cells in culture is not required to improve their adipogenesis.

1. A method of isolating and enriching mesenchymal stem cells (MSCs) comprising:
(a) treating a tissue sample comprising cells and extracellular matrix with an amount of a collagenase that is sufficient to free MSCs from the extracellular matrix in a liquid medium; and
(b) isolating a fraction of the medium containing MSCs.

2. The method according to claim 1 wherein the tissue sample is from bone marrow.

3. The method according to claim 2 wherein the sample is a punch biopsy from the pelvis, sternum or femur.

4. The method according to claim 1, wherein the tissue sample comprises the floating fat fraction (FFF).

5. The method according to claim 1, wherein the tissue sample is from a tissue selected from the group comprising a bone aspirate, synovium and fat pads.

6. The method according to claim 1, wherein the sample is placed in buffer comprising 0.25% collagenase.

7. The method according to claim 1, wherein the sample is treated with collagenase for 3-4 hours.

8. The method according to claim 1, wherein the fraction containing MSCs is obtained by removing any solids and centrifuging the liquid medium to isolate a fraction comprising single cells.

9. The method according to claim 1, wherein step (a) comprises two separate collagenase digestions.

10. The method according to claim 1, wherein step (a) further comprises a hyaluronidase digestion.

11. The method according to claim 9, wherein the tissue sample is bone and the MSCs are for use in bone repair.

12. The method according to claim 1, wherein step (b) further comprises a further enrichment step.

13. The method according to claim 12 wherein the further enrichment step utilizes magnetic beads.

14. The method according to claim 12 wherein the further enrichment step involves FACS sorting.

15. The method according to claim 12, wherein the further enrichment step is based on isolating MSCs with a phenotype characterized by one of the markers identified in Table 1

16. The method according to claim 15 wherein the phenotype is CD45^{low}LNGFR⁺, CD45^{low}D7-FIB⁺LNGFR⁺ or CD45^{low}D7-FIB⁺.

17. Isolated mesenchymal stem cells (MSC) enriched according to the method of claim 1.

18. Isolated mesenchymal stem cells (MSC) according to claim 17 for use as a medicament.

19. A medicament according to claim 18 for use in cell therapy.

20. The medicament according to claim 18 wherein the MSC is genetically manipulated for use in gene therapy.

21. A kit for enriching mesenchymal stem cells (MSC) comprising a collagenase.

22. A method of performing cell therapy on a subject in need of such therapy comprising:
(a) obtaining a tissue sample comprising MSCs and extracellular matrix from said subject;
(b) treating said tissue sample with an amount of a collagenase that is sufficient to free MSCs from the extracellular matrix in a liquid medium;
(c) isolating a fraction of the medium containing MSCs; and
(d) re-introducing said fraction into said subject at a body site that permits said subject to benefit from said cell therapy.

23. A method of performing cell therapy on a subject in need of such therapy comprising:
(a) obtaining a tissue sample comprising MSCs and extracellular matrix from a cadaver or donor;
(b) treating said tissue sample with an amount of a collagenase that is sufficient to free MSCs from the extracellular matrix in a liquid medium;
(c) isolating a fraction of the medium containing MSCs; and
(d) introducing said fraction into said subject at a body site that permits said subject to benefit from said cell therapy.

24. The method according to claim 22 comprising the further step of genetically manipulating the MSC.

25. The method according to claim 22, wherein the cell therapy is for the purpose of clinically managing conditions selected from the group comprising: bone repair, cartilage repair, skeletal muscle repair, tendon repair, ligament repair, meniscus repair, cardiac muscle repair, spinal cord injury, Parkinson's diseases and other neurodegenerative diseases, immune-mediated tissue rejection, graft-versus-host disease, rheumatoid arthritis, regeneration of fatty material, vascular repair or ischaemic vascular lesions or osteoporosis.

26. The method according to claim 22 for repairing bone.

27. The method according to claim 22 for repairing cartilage.

28. The method according to claim 22 for treating osteoarthritis.

29. The method according to claim 22 for treating osteoporosis.

30. (canceled)
